(19) United States
(12) Patent Application Publication
(10) Pub. No.: US 2025/0201377 A1
(43) Pub. Date: Jun. 19, 2025

(54) OPTIMIZATION OF DOSING AND PRESCRIBING OF MEDICATION

(71) Applicant: MannKind Corporation, Danbury, CT (US)

(72) Inventors: Preetaman Kaur Wadhwa, Thousand Oaks, CA (US); Benjamin Harris, Hermosa Beach, CA (US); Emilie Balkman, Atlantic Beach, FL (US); Krystal McCarty, Monrovia, CA (US)

(21) Appl. No.: 18/847,985

(22) PCT Filed: Mar. 15, 2023
(86) PCT No.: PCT/US2023/064406

§ 371 Date: Sep. 17, 2024

Related U.S. Application Data
(60) Provisional application No. 63/321,595, filed on Mar. 18, 2022.

(51) Int. Cl. G16H 20/13

(52) U.S. Cl. CPC G16H 20/13

(57) ABSTRACT

A method of treating a patient with diabetes is provided. The method includes determining a number of powdered insulin units required for each different meal in a day, rounding the number of powdered insulin units up to a nearest predetermined powdered insulin dose for each of the different meals, determining a number and capacity of powdered insulin cartridges to achieve the nearest predetermined powdered insulin doses for each of the different meals, multiplying the number of powdered insulin cartridges of each capacity for each different meal by a number of the different meals in a prescription period, determining a number of boxes of powdered insulin cartridges of each capacity for the prescription period, and prescribing the determined number of boxes of powdered insulin cartridges of each capacity.

### RELATED APPLICATIONS

This application claims the benefit of U.S. Provisional patent application 63/321,595 filed Mar. 18, 2022, the entire contents of which are incorporated by reference herein.

### FIELD

The present disclosure relates to dosage determinations in the treatment of a disease.

### BACKGROUND

Previously, subcutaneous (SC) injection was the only route of delivering insulin for self-administration by patients commercially available. However, SC insulin administration does not lead to optimal pharmacodynamics for the administered insulin because absorption into the blood does not mimic the prandial physiologic insulin secretion pattern. Growing experience with the use of powdered insulin demonstrates that its unique kinetic profile is beneficial in improving glycemic control. However, confusion exists regarding how to start a patient on powdered insulin, at what dose and with what cartridges. Furthermore, it may be unclear which boxes of powdered insulin cartridges of each capacity to prescribe in order to meet a patient's needs. As a result, a patient may receive suboptimal insulin doses for treatment.

Because of the foregoing shortcomings, there is a need to develop a method and system that optimizes dosages.

### SUMMARY

In order to solve the above shortcomings, methods and systems were developed that allow a patient to transition to a powdered insulin. A patient is able to determine the number of powdered insulin units required for each different meal in a day. Based on this information, the disclosure provides methods and systems for determining and prescribing the number of boxes of powdered insulin cartridges of the appropriate dosages for the time period the patient will be using the powdered insulin. These systems and methods may be used for the treatment of any disease that may be ameliorated or arrested by the administration of powdered insulin.

### BRIEF DESCRIPTION OF THE DRAWINGS

The following figures are included to illustrate certain aspects of the present disclosure and should not be viewed as exclusive embodiments. The subject matter disclosed is capable of considerable modifications, alterations, combinations, and equivalents in form and function, as will occur to one having ordinary skill in the art and having the benefit of this disclosure.

FIG. 1 shows a schematic illustration of a system according to the disclosed embodiments.

FIG. 2 illustrates a flowchart of prescription methods for different types of patients.

FIGS. 3-7 illustrate examples of a web based user interface for implementing embodiments of the present disclosure.

FIGS. 8-10 illustrate examples of a mobile based user interface for implementing embodiments of the present disclosure.

### DETAILED DESCRIPTION

Aspects of the present disclosure are directed to improved methods of treating a patient with diabetes that calculates a number of boxes of powdered insulin cartridges of different capacities to be prescribed for a prescription period.

Aspects of the present disclosure are directed to methods of treating a patient with diabetes. A method includes determining a number of powdered insulin units required for each different meal in a day, rounding the number of powdered insulin units up to a nearest predetermined powdered insulin dose for each of the different meals, determining a number and capacity of powdered insulin cartridges to achieve the nearest predetermined powdered insulin doses for each of the different meals, multiplying the number of powdered insulin cartridges of each capacity for each different meal by a number of the different meals in a prescription period, determining a number of boxes of powdered insulin cartridges of each capacity for the prescription period, and prescribing the determined number of boxes of powdered insulin cartridges of each capacity. The type of boxes and/or the number of boxes of powdered insulin cartridges of each capacity to be prescribed for the prescription period will be determined based on the nature of the diabetes.

In some embodiments, the rounding of the number of powdered insulin units is down, unless the rounding would produce zero units.

In some embodiments, the predetermined powdered insulin cartridge capacities include 2, 4, 8, 12, 16, 20, and/or 24 units.

In some embodiments, the predetermined powdered insulin cartridge capacities include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47 and/or 48 units or any suitable dose for a therapy of treating the patient with diabetes.

In some embodiments, the prescription period includes 30, 60, 90, or 120 days.

In some embodiments, the prescription period includes 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175 or 180 days.

In some embodiments, a box of powdered insulin cartridges includes 60 or 90 cartridges.

In some embodiments, a box of powdered insulin cartridges includes 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175 or 180 cartridges or any suitable number for a therapy of treating the patient with diabetes.

In some embodiments, the powdered insulin is in a composition comprising human insulin, fumaryl diketopiperazine and polysorbate 80.

In some embodiments, the powdered insulin is Afrezza®.

In some embodiments, the powdered insulin is Exubera®.

In some embodiments, the diabetes includes one or more of the following diseases: type 1 diabetes, type 2 diabetes, gestational diabetes, latent autoimmune diabetes in adults (LADA), maturity-onset diabetes of the young (MODY) or cystic fibrosis-related diabetes (CFRD).

Another aspect of the present disclosure is directed to systems that include a computing apparatus having a processor and a memory with computer readable program code. The processor under control of the computer readable program code is configured to aid in determining a required number of boxes of powdered insulin cartridges of different capacities for a prescription period by receiving an input comprising a number of insulin units required for each different meal in a day, rounding the number of insulin units up to a nearest predetermined powdered insulin dose for each of the different meals, determining a number and capacity of powdered insulin cartridges to achieve the nearest predetermined powdered insulin doses for each of the different meals, multiplying the number of powdered insulin cartridges of each capacity for each different meal by a number of the different meals in a prescription period, and determining a number of boxes of powdered insulin cartridges of each capacity to be prescribed for the prescription period. The type of boxes and/or the number of boxes of powdered insulin cartridges of each capacity to be prescribed for the prescription period will be determined based on the nature of the disease.

In some embodiments, the rounding of the number of powdered insulin units is down, unless the rounding would produce zero units.

In some embodiments, the predetermined powdered insulin cartridge capacities include 2, 4, 8, 12, 16, 20, and/or 24 units.

In some embodiments, the predetermined powdered insulin cartridge capacities include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47 and/or 48 units or any suitable dose for a therapy of treating a patient with a disease.

In some embodiments, the prescription period includes 30, 60, 90, or 120 days.

In some embodiments, the prescription period includes 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175 or 180 days.

In some embodiments, a box of powdered insulin cartridges includes 60 or 90 cartridges.

In some embodiments, a box of powdered insulin cartridges includes 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175 or 180 cartridges or any suitable number for a therapy of treating a patient with a disease.

In some embodiments, the powdered insulin is in a composition comprising human insulin, fumaryl diketopiperazine and polysorbate 80.

In some embodiments, the powdered insulin is Afrezza®.

In some embodiments, the powdered insulin is Exubera®.

In some embodiments, the powdered insulin is used in the treatment of diabetes, and the diabetes includes one or more of the following diseases: type 1 diabetes, type 2 diabetes, gestational diabetes, latent autoimmune diabetes in adults (LADA), maturity-onset diabetes of the young (MODY) or cystic fibrosis-related diabetes (CFRD).

Another aspect of the present disclosure is directed to methods of treating a patient with a disease, wherein the symptoms of the disease are ameliorated or arrested by the administration of insulin. The method includes determining a number of powdered insulin units required for each different meal in a day, rounding the number of powdered insulin units up to a nearest predetermined powdered insulin dose for each of the different meals, determining a number and capacity of powdered insulin cartridges to achieve the nearest predetermined powdered insulin doses for each of the different meals, multiplying the number of powdered insulin cartridges of each capacity for each different meal by a number of the different meals in a prescription period, determining a number of boxes of powdered insulin cartridges of each capacity for the prescription period, and prescribing the determined number of boxes of powdered insulin cartridges of each capacity. The type of boxes and/or the number of boxes of powdered insulin cartridges of each capacity to be prescribed for the prescription period will be determined based on the nature of the disease.

In some embodiments, the rounding of the number of powdered insulin units is down, unless the rounding would produce zero units.

In some embodiments, the predetermined powdered insulin cartridge capacities include 2, 4, 8, 12, 16, 20, and/or 24 units.

In some embodiments, the predetermined powdered insulin cartridge capacities include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47 and/or 48 units or any suitable dose for a therapy of treating the patient with the disease.

In some embodiments, the prescription period includes 30, 60, 90, or 120 days.

In some embodiments, the prescription period includes 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175 or 180 days.

In some embodiments, a box of powdered insulin cartridges includes 60 or 90 cartridges.

In some embodiments, a box of powdered insulin cartridges include 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175 or 180 cartridges or any suitable number for a therapy of treating the patient with the disease.

In some embodiments, the powdered insulin is in a composition comprising human insulin, fumaryl diketopiperazine and polysorbate 80.

In some embodiments, the powdered insulin is Afrezza®.

In some embodiments, the powdered insulin is Exubera®.

In some embodiments, the disease includes one or more of the following diseases: type 1 diabetes, type 2 diabetes, gestational diabetes, diabetic ketoacidosis (DKA), hyperosmolar hyperglycemic state (HHS), latent autoimmune diabetes in adults (LADA), maturity-onset diabetes of the young (MODY), insulin resistance syndrome (IRS), lipodystrophy, polycystic ovary syndrome (PCOS), hyperkalemia or cystic fibrosis-related diabetes (CFRD).

It should be understood that at least in some embodiments, powdered insulin may also refer to inhaled insulin.

While the disclosed embodiments are described in the context of powdered insulin, it should be understood that the disclosed embodiments may be applicable to any medicament.

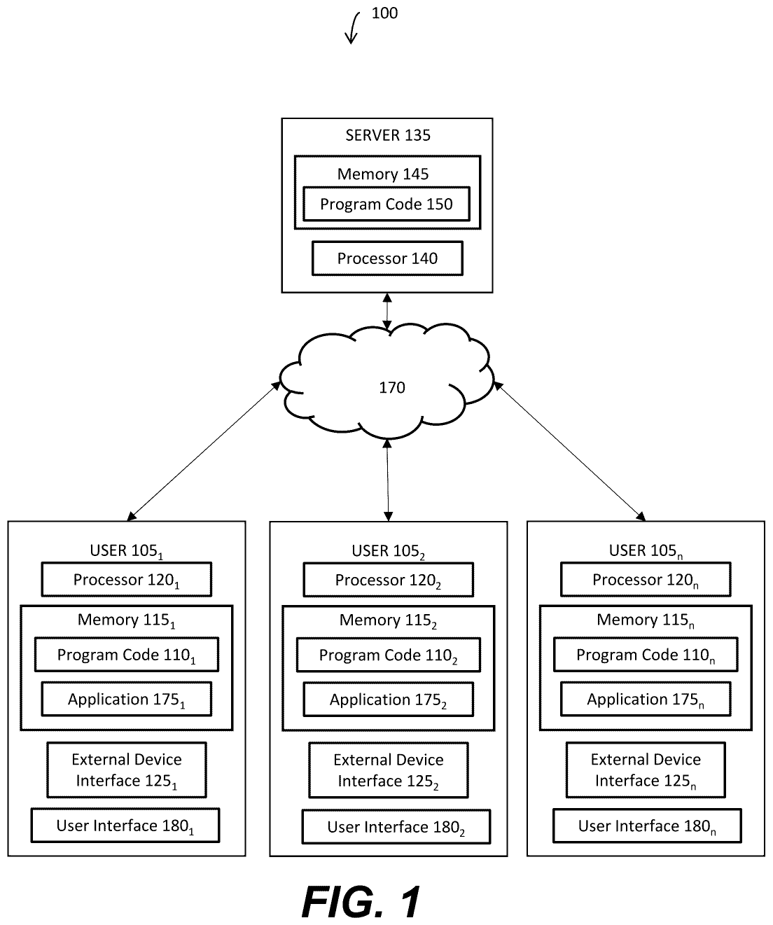

FIG. 1 shows a schematic illustration of system 100 configured to provide the method of treating a patient with diabetes. One or more users may access the system 100 through user terminals 105₁-105ₙ. Users of the disclosed system may include physicians and any authorized medical practitioners.

The user terminals 105₁-105ₙ may operate an application 175₁-175ₙ that provides access to a server 135 providing the method, or in some embodiments, the application 175₁-175ₙ may be self-contained in each user terminal 105₁-105ₙ. The user terminals 105₁-105ₙ may each include an external device interface 125₁-125ₙ that may include a web browser. The user terminals 105₁-105ₙ may each further include a user interface 180₁-180ₙ that may include one or more input and output devices, for example, a keyboard, touch screen or voice control module, and a display, virtual reality or augmented reality device.

Each user may access the application 175₁-175ₙ at anytime from anywhere through any suitable user terminal, which may include for example, a desktop computer, a laptop, a tablet, a mobile phone, or any other computing device capable of performing the functions of the disclosed embodiments. It should be understood that a user may utilize more than one computing device and more than one type of computing device to access the career planning and project collaboration tools. As another example, a user may use a mobile phone as a user terminal at one point in time to access the system and later may use a tablet as a user terminal.

Each user terminal 105₁-105ₙ may include computer readable program code 110₁-110ₙ stored on at least one non-transitory computer readable medium for carrying out and executing the processes described herein. In at least one embodiment, the computer readable program code 110₁-110ₙ may invoke or operate the application 175₁-175ₙ for providing, in combination with other components of the system 100, the method of treating a patient with diabetes. The computer readable medium may be memories 115₁-115ₙ, and in alternate aspects, the computer readable program code 110₁-110ₙ may be stored in memories external to, or remote from, user terminals 105₁-105ₙ. Memories 115₁-115ₙ may include magnetic media, semiconductor media, optical media, or any media which is readable and executable by a computer. Each user terminal 105₁-105ₙ may also include a processor 120₁-120ₙ for executing the computer readable program code 110₁-110ₙ. Each user terminal 105₁-105ₙ may also include at least one interface 125₁-125ₙ to external devices as d above.

The system 100 may include one or more servers 135, each with a processor 140, and memory 145 storing computer program code 150 for generally operating the server 135 to provide the method of treating a patient with diabetes described herein. In some embodiments, the one or more servers 135 may be implemented by a cloud computing service, and the method of treating a patient with diabetes may be provided in the form of software as a service (SaaS). The various components of the system 100, including the user terminals 105₁-105ₙ and one or more servers 135, may communicate over a network 170.

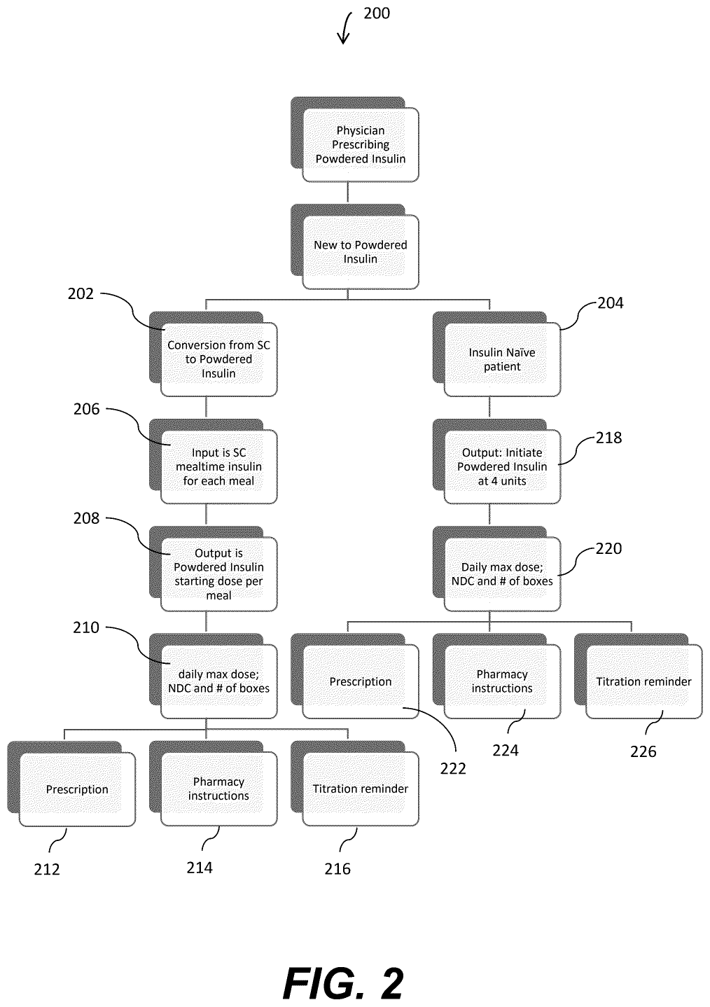

FIG. 2 illustrates a flowchart of an overview 200 of the operation of the disclosed application for patients currently using insulin 202 in an injectable form and insulin naïve patients 204. For patients currently using insulin 202, the application includes inputting an amount of subcutaneous insulin currently used for each meal 206. The application then outputs a starting dose of powdered insulin for each meal 208, and calculates a daily maximum dose of powdered insulin, the number of types of cartridges, corresponding stock numbers (NDC) and number of boxes of each type of cartridge 210. The application then operates to generate a prescription 212, pharmacy instructions 214, and a titration reminder 216.

For insulin naïve patients 204, the application initiates a starting dose of 4 units of powdered insulin for each meal 218, and calculates a daily maximum dose of powdered insulin, the number of types of cartridges, corresponding stock numbers (NDC) and number of boxes of each type of cartridge 220. The application then operates to generate a prescription 222, pharmacy instructions 224, and a titration reminder 226.

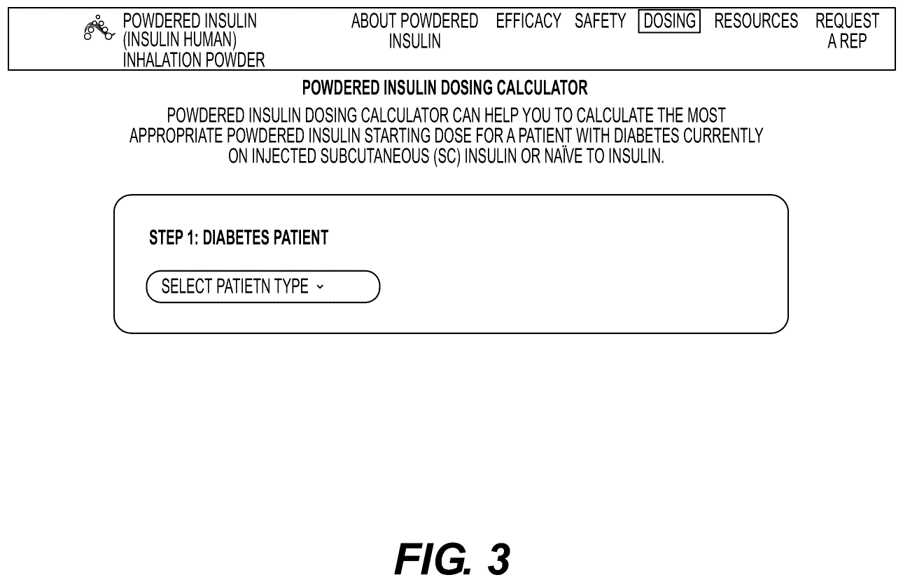

FIGS. 3-7 illustrate examples of a web based user interface for implementing embodiments of the present disclosure.

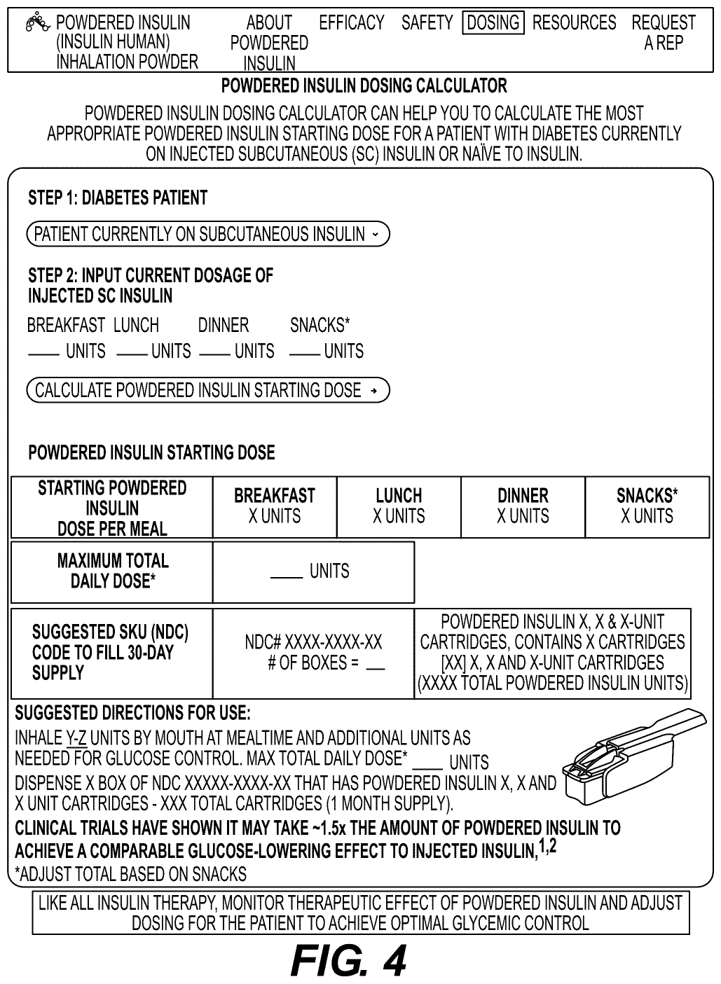

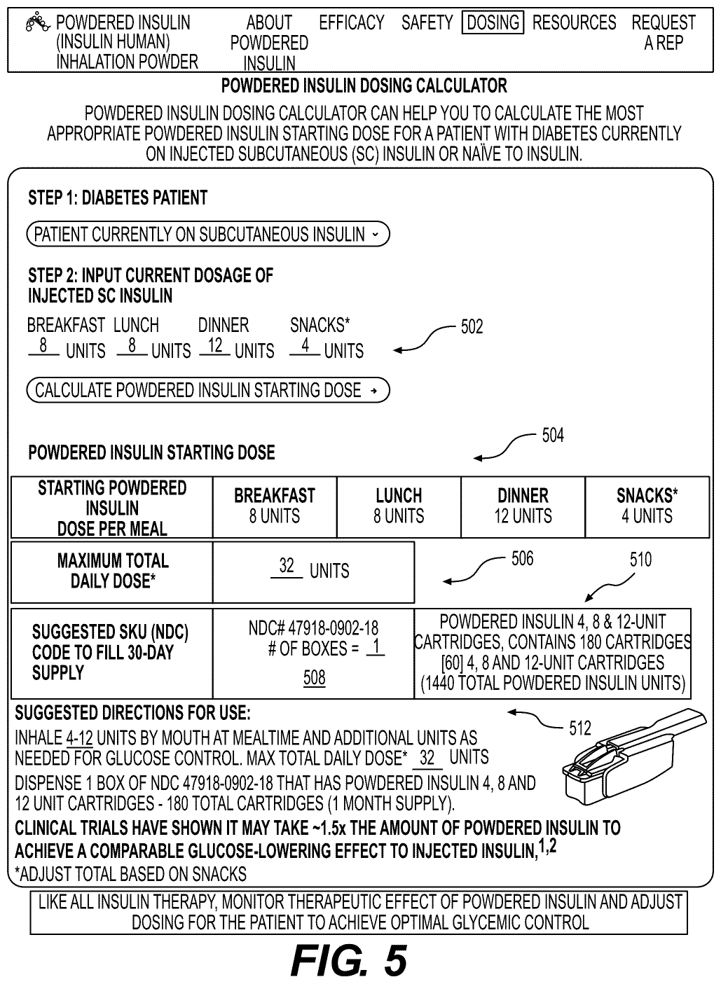

As shown in FIG. 3, the user interface may allow a user to select a patient type as being either a patient currently on subcutaneous (SC) insulin or an insulin naïve patient. For example, a user may select a patient currently on subcutaneous insulin as shown in FIG. 4. The application may then present the user with the ability to input current dosages of subcutaneous insulin for each meal (i.e., breakfast, lunch, and dinner) and snack(s). Upon receiving the current dosages of subcutaneous insulin for each meal and snack(s) from the user, the application proceeds to calculate starting dosages of powdered insulin for each meal and snack 504, as well as a maximum total dose 506, number of boxes of cartridges 508, and the types of cartridges 510, as shown in FIG. 5. The application may also generate suggested directions for use 512. For example, for the input starting dosage of powdered insulin, breakfast may have 8 units, lunch 8 units, dinner 12 units and snacks 4 units. In this embodiment, the maximum total daily dose (this value may be adjusted based on the number of snacks) may be 32 units. The number of boxes of cartridges may be one. The types of cartridges may be 4, 8 and 12 unit cartridges. The suggested directions for use may state, for example, inhale 4-12 units by mouth at mealtime and additional units as needed for glucose control. The instructions may further state, for example, that the maximum total daily dose (this value may be adjusted based on the number of snacks) is 16 units. And the instructions may also state, for example, the following:
i) name of the medication is Afrezza®.
ii) Dispense 1 box of NDC 47918-0902-18 (suggested SKU code to fill 30-day supply) that has Afrezza® 4, 8 and 12 unit cartridges—180 total cartridges (1 month supply).
iii) Clinical trials have shown it may take the amount of Afrezza® to achieve a comparable glucose-lowering effect to injected insulin. (Bode B W, McGill J B, Lorber D L, Gross J L, Chang P C, Bregman D B. Inhaled Technosphere insulin compared with injected prandial insulin in type 1 diabetes: a randomized 24-week trial. Diabetes Care. 2015; 38 (12): 2266-2273; 2. Akturk H K, Snell-Bergeon J K, Rewers A, et al. Improved postprandial glucose with inhaled Technosphere insulin compared with insulin aspart in patients with type 1 diabetes on multiple daily injections: the STAT study. Diabetes Technol Ther. 2018; 20 (10): 639-647).
iv) Like all insulin therapy, monitor therapeutic effect of Afrezza® and adjust dosing for the patient to achieve optimal glycemic control.

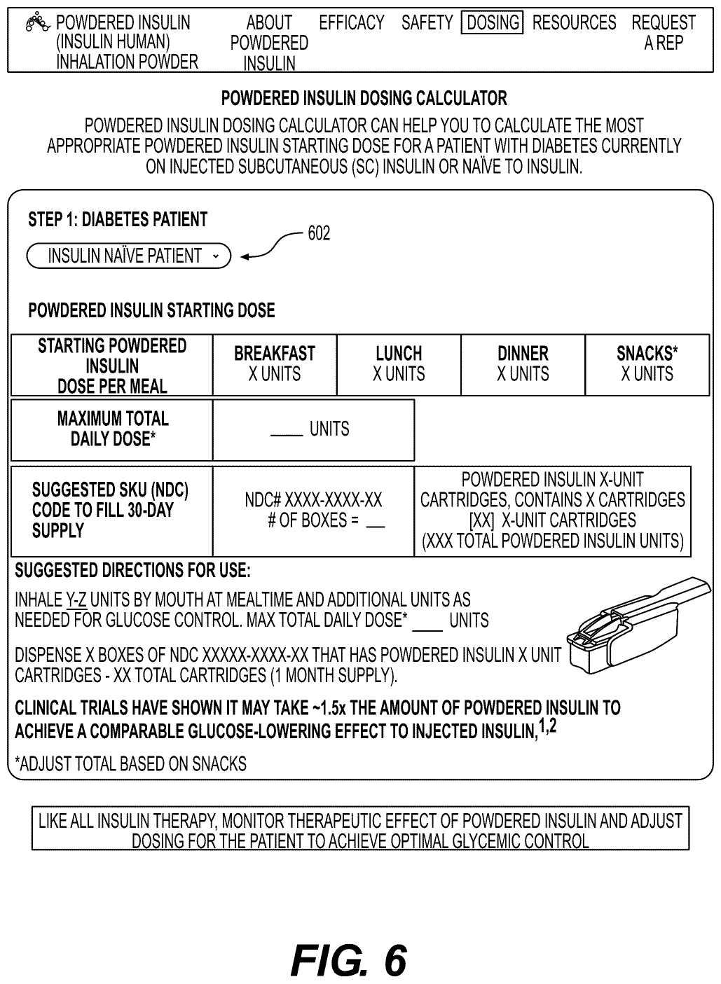

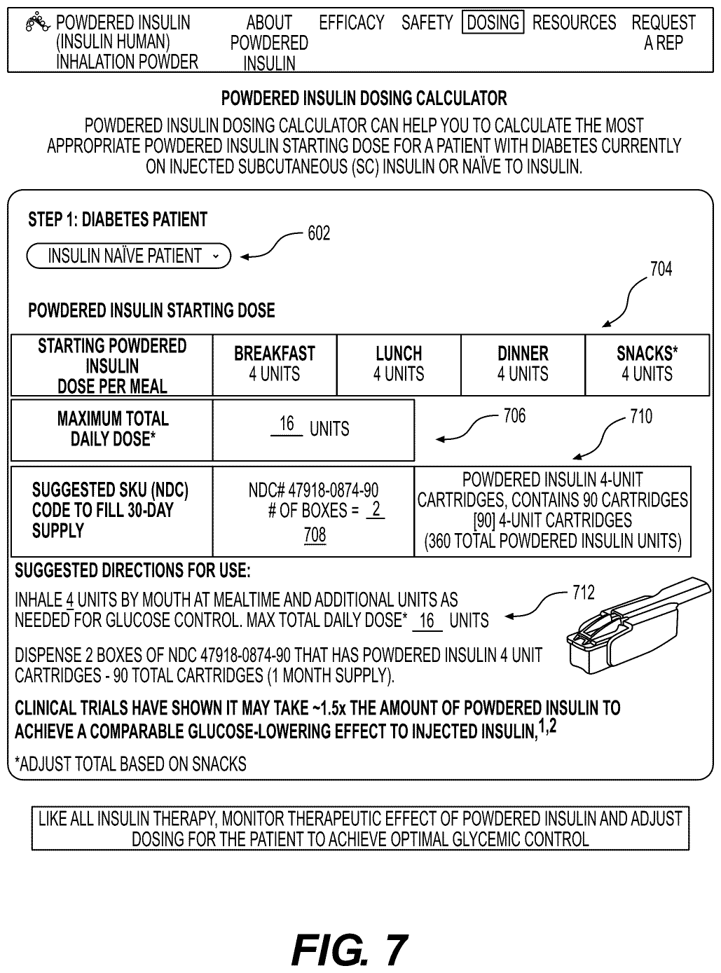

As discussed above, the application allows a user to select an insulin naïve patient 602 as shown in FIG. 6. The application may then present the user with a predetermined starting dose of powdered insulin for each meal and snack 704, a calculated maximum total dose 706, a number of boxes of cartridges 708, and the types of cartridges 710 as shown in FIG. 7. The application may also generate suggested directions for use 712. For example, for the input starting dosage of powdered insulin, breakfast may have 4 units, lunch 4 units, dinner 4 units and snacks 4 units. In this embodiment, the maximum total daily dose (this value may be adjusted based on the number of snacks) may be 16 units. The number of boxes of cartridges may be two. The types of cartridges may be 4 unit cartridges. The suggested directions for use may state, for example, inhale 4 units by mouth at mealtime and additional units as needed for glucose control. The instructions may further state, for example, that the maximum total daily dose (this value may be adjusted based on the number of snacks) is 16 units. And the instructions may also state, for example, the following:
i) name of the medication is Afrezza®.
ii) Dispense 2 boxes of NDC 47918-0874-90 (suggested SKU code to fill 30-day supply) that has Afrezza® 4 unit cartridges—90 total cartridges (1 month supply).
iii) Clinical trials have shown it may take the amount of Afrezza® to achieve a comparable glucose-lowering effect to injected insulin. (Bode B W, McGill J B, Lorber D L, Gross J L, Chang P C, Bregman D B. Inhaled Technosphere insulin compared with injected prandial insulin in type 1 diabetes: a randomized 24-week trial. Diabetes Care. 2015; 38 (12): 2266-2273; 2. Akturk H K, Snell-Bergeon J K, Rewers A, et al. Improved postprandial glucose with inhaled Technosphere insulin compared with insulin aspart in patients with type 1 diabetes on multiple daily injections: the STAT study. Diabetes Technol Ther. 2018; 20 (10): 639-647).
iv) Like all insulin therapy, monitor therapeutic effect of Afrezza® and adjust dosing for the patient to achieve optimal glycemic control.

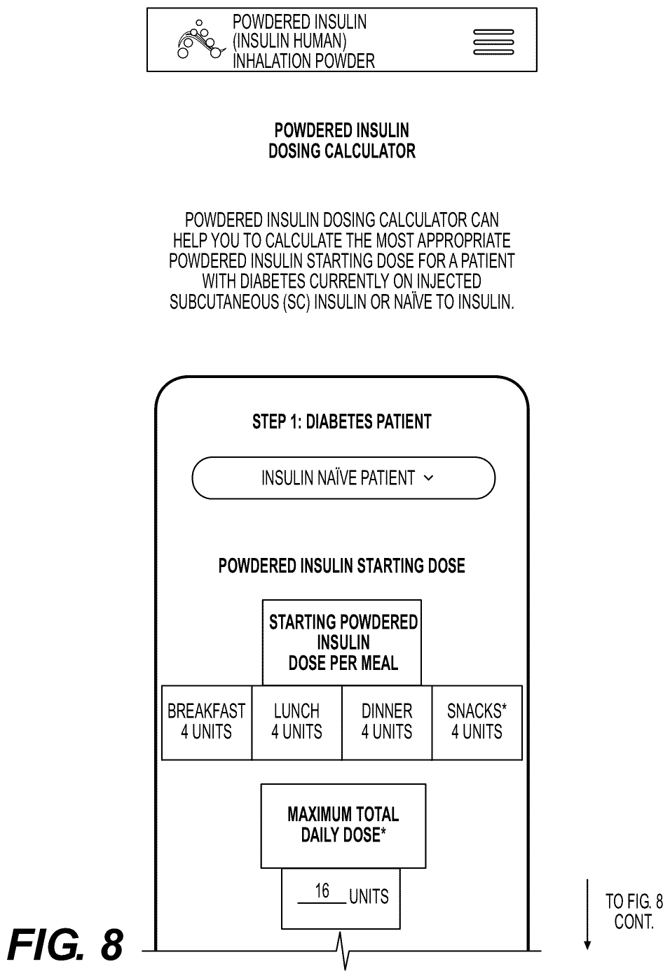

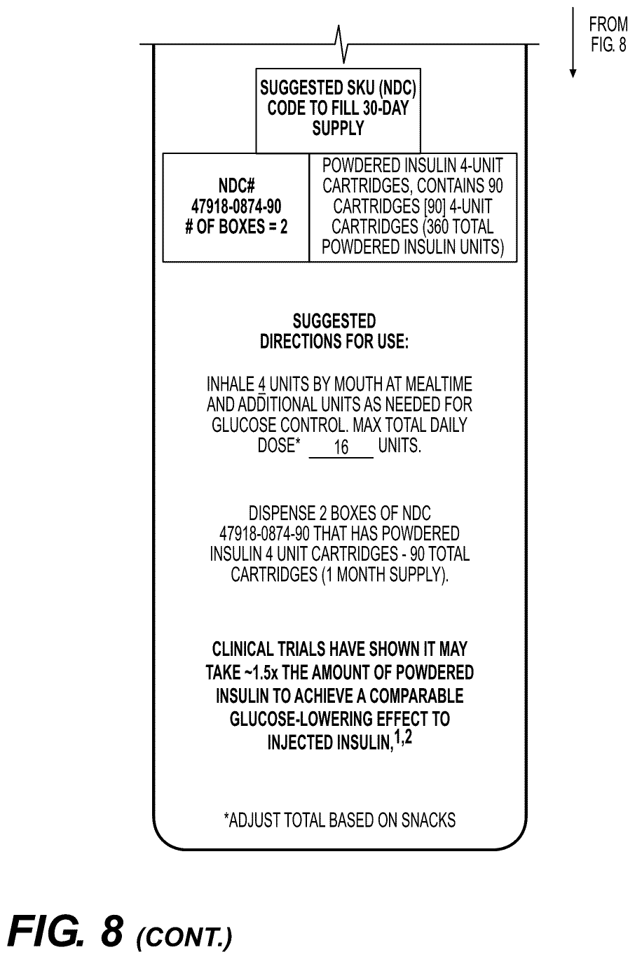

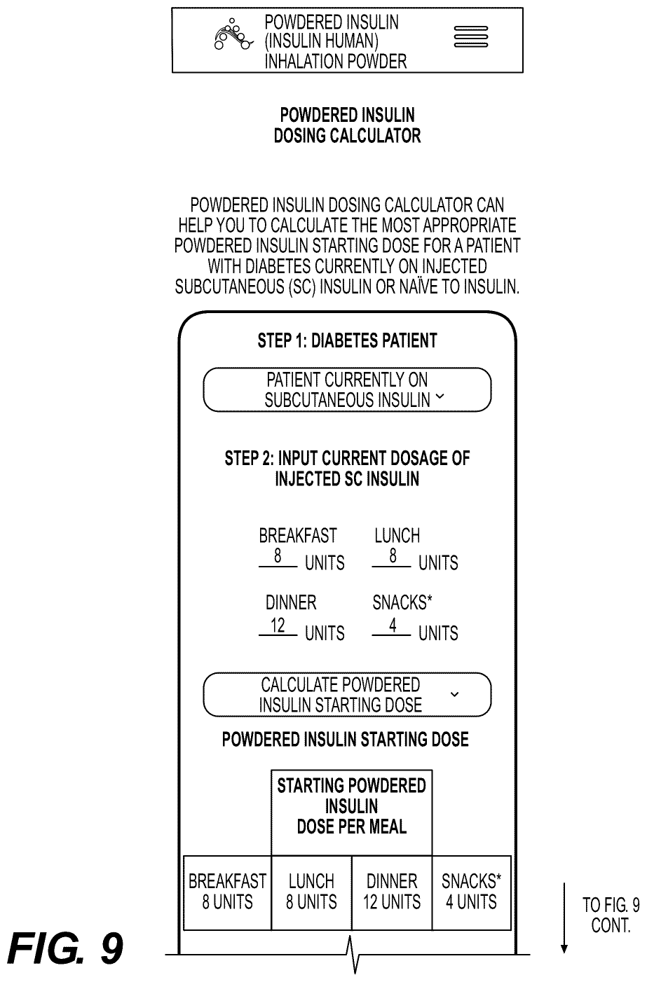

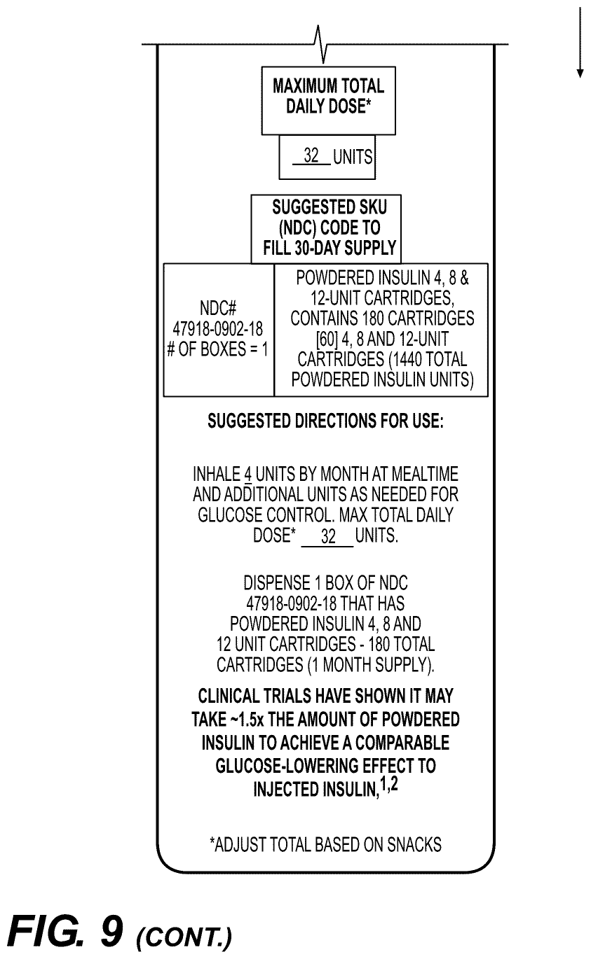

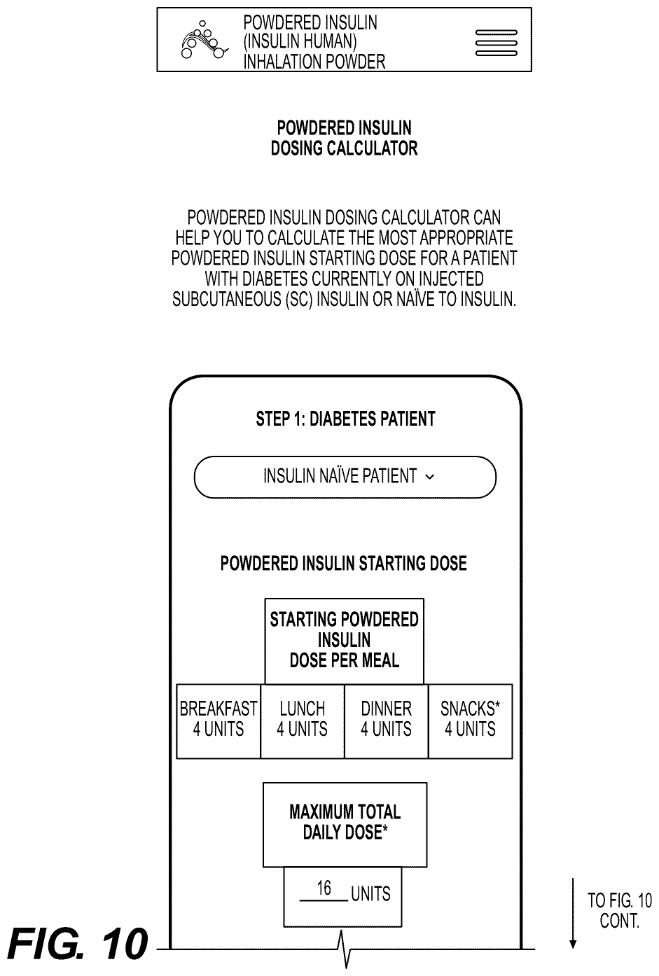

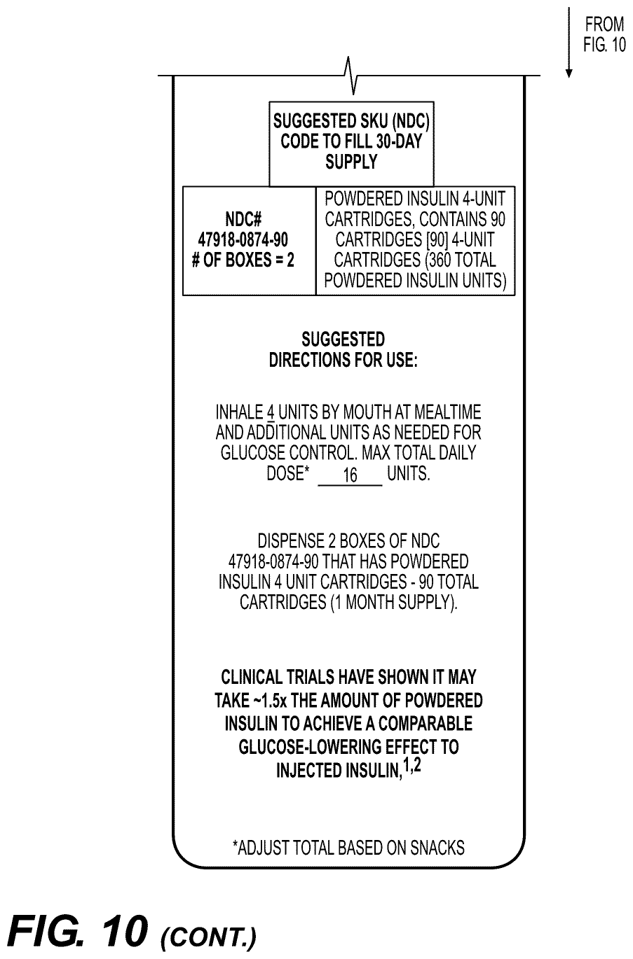

FIGS. 8-10 illustrate examples of a mobile based user interface for implementing embodiments of the present disclosure in the same manner as illustrated in FIGS. 3-7.

The application may calculate the powdered insulin starting dose by rounding the number of subcutaneous insulin units for each meal to a corresponding number of available powdered insulin units as packaged. For example, powdered insulin may be packaged in 4, 8, and 12 unit cartridges. In some embodiments, powdered insulin may be packaged in 2, 4, 8, 12, 16, 24, 28, 32, 36, 40, 44, 48, 52 or any number of units suitable for therapy. As a result, any fractional value of between 0-2 subcutaneous insulin units may be rounded up to 2 powdered insulin units, any fractional value of between 3 and 4 subcutaneous insulin units may be rounded up to 4 powdered insulin units, any fractional value of between 5 and 8 subcutaneous insulin units may be rounded up to 8 powdered insulin units, any fractional value of between 9 and 16 subcutaneous insulin units may be rounded up to 16 powdered insulin units, etc.

The maximum daily dose may be calculated by adding the individual doses for the meals, breakfast, lunch, dinner and snack (BDLS). The number of doses required for a particular prescription period may be calculated by multiplying the maximum daily dose by the number of days in the prescription period.

The proper stock numbers for the types and numbers of different dosage cartridges may be determined by using the following calculations based on the BDLS doses:
for BDLS doses of 4 or 4 and 0, then 4 unit cartridges may be used;
for BDLS doses of 8 or 8 and 0, then 8 unit cartridges may be used;
for BDLS doses of 12 or 12 and 0, then 12 unit cartridges may be used;
for BDLS doses of 4 and 8, or 4, 8, and 0, then 4 and 8 unit cartridges may be used;
if either BDLS are less than 12, then 4 and 8 unit cartridges may be used;
for BDLS doses of 8 and 12, or 8, 12, and 0, then 8 and12 unit cartridges may be used; and
if any of breakfast, lunch, dinner or snack are greater than 12, then a titration collection of 4, 8, and 12 unit cartridges may be used.

While the calculated dosages may be advantageous for initial prescriptions, the powdered insulin dosages may be further adjusted based on a patient's metabolic needs, reaction to different dosage levels, and glycemic control goals. Dosage adjustments may further be based on changes in physical activity, meal patterns, renal or hepatic function, or during illness.

The user interfaces for the foregoing embodiments may be either web based or mobile based. The interfaces may include text such as: Afrezza® Dosing Calculator can help you to calculate the most appropriate Afrezza® starting dose for a patient with diabetes currently on injected subcutaneous (SC) insulin or naïve to insulin. The interfaces may also have tabs in which the user may access, for example, efficacy, safety, dosing information about Afrezza® (or whatever medication is being used). Afrezza® is used in the instant application soley as an example. Other medications may be used in the foregoing embodiments, or in a modified embodiment that will become apparent to those of ordinary skill in the art upon reading the foregoing description.

As used herein, the term “individual”, “patient”, or “subject” used interchangeably, refers to any animal, including mammals, preferably mice, rats, other rodents, rabbits, dogs, cats, swine, cattle, sheep, horses, or primates, and most preferably humans.

As used herein the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member may be referred to and claimed individually or in any combination with other members of the group or other elements found herein. It is anticipated that one or more members of a group may be included in, or deleted from, a group for reasons of convenience or patentability. When any such inclusion or deletion occurs, the specification is deemed to contain the group as modified thus fulfilling the written description of all Markush groups used in the appended claims.

Certain embodiments of this disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the disclosure so claimed are inherently or expressly described and enabled herein. Claim language reciting “at least one of” a set indicates that one member of the set or multiple members of the set satisfy the claim.

Furthermore, references to patents and printed publications may have been made in this specification. Each of the above-cited references and printed publications are individually incorporated herein by reference in their entirety.

In closing, it is to be understood that the embodiments of the disclosure disclosed herein are illustrative of the principles of the present disclosure. Other modifications that may be employed are within the scope of the disclosure. Thus, by way of example, but not of limitation, alternative configurations of the present disclosure may be utilized in accordance with the teachings herein. Accordingly, the present disclosure is not limited to that precisely as shown and described.

We claim:

1. A method of treating a patient with diabetes comprising:
determining a number of powdered insulin units required for each different meal in a day;
rounding the number of powdered insulin units up to a nearest predetermined powdered insulin dose for each of the different meals;
determining a number and capacity of powdered insulin cartridges to achieve the nearest predetermined powdered insulin doses for each of the different meals;
multiplying the number of powdered insulin cartridges of each capacity for each different meal by a number of the different meals in a prescription period;
determining a number of boxes of powdered insulin cartridges of each capacity for the prescription period; and
prescribing the determined number of boxes of powdered insulin cartridges of each capacity.

2. The method of claim 1, wherein the predetermined powdered insulin cartridge capacities comprise 2, 4, 8, 12, 16, 20, and/or 24 units.

3. The method of claim 1, wherein the prescription period comprises 30, 60, 90, or 120 days.

4. The method of claim 1, wherein a box of powdered insulin cartridges comprises 60 or 90 cartridges.

5. The method of claim 1, wherein the powdered insulin is in a composition comprising human insulin, fumaryl diketopiperazine and polysorbate 80.

6. The method of claim 1, wherein the diabetes comprises one or more of the diseases selected from the group consisting of type 1 diabetes, type 2 diabetes, gestational diabetes, latent autoimmune diabetes in adults (LADA), maturity-onset diabetes of the young (MODY) and cystic fibrosis-related diabetes (CFRD).

7. A system comprising a computing apparatus having a processor and a memory with computer readable program code, the processor under control of the computer readable program code configured to aid in determining a required number of boxes of powdered insulin cartridges of different capacities for a prescription period by:
receiving an input comprising a number of insulin units required for each different meal in a day;
rounding the number of insulin units up to a nearest predetermined powdered insulin dose for each of the different meals;
determining a number and capacity of powdered insulin cartridges to achieve the nearest predetermined powdered insulin doses for each of the different meals;
multiplying the number of powdered insulin cartridges of each capacity for each different meal by a number of the different meals in a prescription period; and
determining a number of boxes of powdered insulin cartridges of each capacity to be prescribed for the prescription period.

8. The system of claim 7, wherein the predetermined powdered insulin cartridge capacities comprise 2, 4, 8, 12, 16, 20, and/or 24 units.

9. The system of claim 7, wherein the prescription period comprises 30, 60, 90, or 120 days.

10. The system of claim 7, wherein a box of powdered insulin cartridges comprises 60 or 90 cartridges.

11. The system of claim 7, wherein the powdered insulin is in a composition comprising human insulin, fumaryl diketopiperazine and polysorbate 80.

12. The system of claim 7, wherein the powdered insulin is used in the treatment of diabetes, and
wherein the diabetes comprises one or more of the diseases selected from the group consisting of type 1 diabetes, type 2 diabetes, gestational diabetes, latent autoimmune diabetes in adults (LADA), maturity-onset diabetes of the young (MODY) and cystic fibrosis-related diabetes (CFRD).

13. A method of treating a patient with a disease, wherein the symptoms of the disease are ameliorated or arrested by the administration of insulin, comprising:
determining a number of powdered insulin units required for each different meal in a day;
rounding the number of powdered insulin units up to a nearest predetermined powdered insulin dose for each of the different meals;
determining a number and capacity of powdered insulin cartridges to achieve the nearest predetermined powdered insulin doses for each of the different meals;
multiplying the number of powdered insulin cartridges of each capacity for each different meal by a number of the different meals in a prescription period;
determining a number of boxes of powdered insulin cartridges of each capacity for the prescription period; and
prescribing the determined number of boxes of powdered insulin cartridges of each capacity.

14. The method of claim 13, wherein the predetermined powdered insulin cartridge capacities comprise 2, 4, 8, 12, 16, 20, and/or 24 units.

15. The method of claim 13, wherein the prescription period comprises 30, 60, 90, or 120 days.

16. The method of claim 13, wherein a box of powdered insulin cartridges comprises 60 or 90 cartridges.

17. The method of claim 13, wherein the powdered insulin is in a composition comprising human insulin, fumaryl diketopiperazine and polysorbate 80.

18. The method of claim 13, wherein the disease comprises one or more of the diseases selected from the group consisting of type 1 diabetes, type 2 diabetes, gestational diabetes, diabetic ketoacidosis (DKA), hyperosmolar hyperglycemic state (HHS), latent autoimmune diabetes in adults (LADA), maturity-onset diabetes of the young (MODY), insulin resistance syndrome (IRS), lipodystrophy, polycystic ovary syndrome (PCOS), hyperkalemia and cystic fibrosis-related diabetes (CFRD).
